(19) United States
(12) Patent Application Publication
(10) Pub. No.: US 2026/0311119 A1
(43) Pub. Date: Oct. 8, 2026

(54) FIRST AID CABINET CONFIGURATION FOR RFID INVENTORY CONTROL AND COMPLIANCE CARD WITH ANGLED OR OFFSET RFID SUPPORT PORTION

(71) Applicant: Acme United Corporation, Shelton, CT (US)

(72) Inventors: Brian S. Olschan, Madison, CT (US); Kathleen Levay, Milford, CT (US); Michael A. Healey, York, ME (US); Janice Fowler, Brandord, CT (US)

(21) Appl. No.: 19/638,308

(22) Filed: Apr. 3, 2026

Related U.S. Application Data
(60) Provisional application No. 63/783,507, filed on Apr. 4, 2025.
(60) Provisional application No. 63/840,798, filed on Jul. 9, 2025.

(51) Int. Cl. G06Q 10/087; G06K 7/10

(52) U.S. Cl. CPC G06Q 10/08726; G06K 7/10297; G06K 7/10415

(57) ABSTRACT

A first aid cabinet configured for RFID inventory control incorporates an RFID reader within the cabinet and a compliance card including an RFID tag in boxes of first aid product stored in the cabinet. The compliance card includes a divider portion positioned between first aid product in the box to divide the first aid product into a primary portion in front of the divider portion and a reserve portion of first aid product behind the divider portion. The compliance card includes an RFID support portion angled or offset from the divider portion, allowing an RFID tag to be positioned between the first aid product and the box so interrogation of the RFID tag is not interfered with by foil packaging or liquids in the first aid products. The compliance card is removed when the primary portion of first aid product is depleted to expose a front side of the divider portion. An inventory control system generates a restocking requisition for the first aid product associated with each compliance card that has been removed from the cabinet.

### BACKGROUND

This disclosure relates generally to first aid cabinet configurations and techniques for ensuring that a first aid cabinet is fully stocked with first aid supplies. More particularly, this disclosure relates to systems which employ radio frequency identification (RFID) tags and automated inventory methods to monitor the contents of first aid cabinets and to order and resupply first aid items.

The Occupational Safety and Health Administration (OSHA) is an agency of the United States federal government tasked with enforcing regulations mandating that employers provide adequate first aid supplies to treat common workplace injuries. Many states also have workplace safety regulations requiring employers to provide access to first aid supplies. Some industries such as construction and manufacturing carry a higher risk of injury, increasing the importance of employee access to first aid supplies. One way for an employer to meet the requirement to provide access to first aid supplies is to position well-marked first aid cabinets in the workplace. The first aid cabinets have contents tailored to specific work environments and must be re-stocked as first aid products are removed or expire. Some large facilities will have many first aid cabinets, making the task of maintaining a stock of first aid products in all the cabinets a labor-intensive task. Many first aid products such as bandages, gauze pads, and ointments are packaged individually, with a pre-determined quantity of the product contained in a box or bin within the enclosure.

U.S. Pat. No. 12,008,630, (hereafter “the '630 patent,” assigned to the applicant of the present disclosure), discloses a cabinet electronic requisition system that uses compliance cards to alert personnel responsible for maintaining first aid cabinets of the need to re-stock a category of first aid product when the quantity of the product reaches a pre-determined minimum quantity. The compliance cards are positioned within a container such as a box so that when a pre-determined quantity of a product is removed from the container, the compliance card is conspicuously visible. The compliance card includes instructions for the card to be removed from the container and either scanned or disposed of. The '630 patent discloses one embodiment of a first aid cabinet where the compliance cards are equipped with an RFID tag, and the cabinet is configured to periodically read all the tags in the cabinet and automatically generate a re-stocking order for a first aid product when the compliance card carrying an RFID tag for the first aid product is not present in the cabinet. The RFID enabled system of the '630 patent substantially automates inventory and ordering functions for maintaining first aid products in cabinets throughout a facility.

To be effective, an RFID enabled system such as that disclosed in the '630 patent needs to reliably activate and read all the RFID tags in the first aid cabinet. The packaging for some first aid products may include metal foil. Other first aid products include viscous liquids or gels such as ointments for the treatment of burns. Foil packaging and liquids can interfere with activation and reading of RFID tags on compliance cards positioned within boxes or bins of first aid products in a first aid cabinet. Boxes or bins of first aid products containing liquids or having foil packaging can interfere with activation and reading of RFID tags within containers of other first aid products in a first aid cabinet.

There is a need for a first aid cabinet and first aid product packaging configured to ensure reliable reading of all RFID tags in a first aid cabinet.

### SUMMARY OF THE INVENTION

A disclosed first aid cabinet may include a metal enclosure defining an interior having a height H, a width W and a depth D, the enclosure including a door and a latch to secure the door in a closed position. A non-metal cabinet enclosure constructed of plastic may also be used. A plastic enclosure may be lined with a metallic layer to contain radio frequency signals within the cabinet. A product support grid constructed of non-conductive material fills a rear portion of the enclosure. The product support grid defines a plurality of rows of compartments and an RFID reader compartment within one of the plurality of rows of compartments. In one embodiment, each row of compartments is offset within the depth D of the enclosure relative to the other rows of compartments. According to aspects of the disclosure, the RFID reader compartment may be positioned between 10% and 20% of the height H above or below a center of the height H of the enclosure. An RFID reader positioned in the RFID reader compartment includes an RFID antenna. The RFID antenna may be positioned forward of a center of the depth D of the enclosure. In one embodiment, the antenna is forward of a center of the depth D of the enclosure by a distance between 10% and 20% of the depth D of the enclosure. In another embodiment the RFID antenna is spaced apart from the door of the enclosure a distance between approximately 10% to 25% of the depth D when the door is in a closed position.

According to aspects of the disclosure, first aid products such as bandages, alcohol wipes, ointments, and the like are provided in boxes or containers received in compartments defined by the product support grid. In this disclosure, the term “box” will be used when discussing containers for first aid products and should be interpreted to include alternatives such as molded plastic containers, bins or bags containing a quantity of the same first aid product. A box of first aid products is received in each compartment, each box including a compliance card having a divider portion and an RFID support portion angled or offset relative to the divider portion. An RFID tag on the RFID support portion is positioned between first aid products in the box and a top, bottom, side, back, or front of the box and the divider portion is arranged between the first aid products to divide the first aid products into a first portion and a second portion. The first portion of first aid products is in front of the divider portion, between the divider portion and an opening in the box or container through which first aid products are removed. The second portion of first aid products is behind the divider portion, defining a predetermined number of the first aid products in the box behind the divider portion of the compliance card. Each box of first aid products includes a front side exposed at a front of the product support grid, the front having an opening through which products can be removed from the box. One example of a box is a paperboard box including a portion defined by perforations that can be removed to define an opening to access first aid products within the box.

The removable portion of the box is removed define an opening in the front side of each box to allow dispensing of first aid product through the opening. As first aid products are removed from the box through the opening, the first portion of first aid products is depleted, exposing the compliance card. A front face of the compliance card is visible through the opening of the box when the last of the first portion of first aid products has been removed. The portion of the compliance card visible through the opening of the box may be brightly colored and includes a conspicuous notice to remove the compliance card to initiate requisition of a replacement box of the first aid products. After the compliance card is removed, the second portion of first aid products remains in the box and available to users of the first aid cabinet during the time necessary to complete the order, delivery and re-stocking of the first aid products. This ensures the first aid cabinet always has at least some of each first aid product. The number of first aid products in each package and the number of first aid products in the first portion in front of the compliance card and in the second portion behind the compliance card are determined according to the expected/actual use of the first aid products and the time period necessary to complete the ordering and restocking of the first aid products.

The compliance card includes an RFID tag positioned between the first aid products and the box or container. The RFID tag is loaded with information including at least the type of first aid product in each box. The RFID reader is configured to periodically read all the RFID tags in the enclosure and compare a list of first aid products in the enclosure with a list of first aid products representing a fully stocked first aid cabinet to identify first aid products associated with a compliance card that has been removed from the first aid cabinet. The RFID tag may also include expiration dates for each box, bag or other container of first aid products, and the requisition system may be configured to automatically order first aid products as they approach their expiration date.

In some embodiments of a compliance card, the divider portion is connected to the RFID support portion along an edge of the divider portion, and the RFID support portion is angled relative to the divider portion. This allows the RFID support portion to be positioned between the first aid product and the box, package, or container while the divider portion is positioned between the first portion and second portion of the first aid products. In an alternative embodiment of a compliance card, the divider portion is connected to the RFID support portion by a connector portion, with the RFID support portion offset from the divider portion and oriented parallel to the divider portion. Because the RFID support portion is connected to the divider portion, removal of the compliance card removes the RFID tag along with the compliance card. In one embodiment of a compliance card with a connector portion, the divider portion is arranged vertically between the first and second portions of first aid products in the box, the connector portion is horizontally positioned at the bottom of the box and the RFID support portion is positioned vertically at the front of the box, beneath the opening through which first aid products are removed from the box. This compliance card configuration and position places the RFID tag at the front of each box where it is reliably detected by the RFID reader.

A requisition system including the disclosed first aid cabinet automatically generates a restocking requisition for the first aid product associated with each compliance card that has been removed from the first aid cabinet.

First aid products in the first aid cabinet may include first aid products comprising a liquid or packaged in packaging including metal foil. The product support grid may be configured to position boxes of first aid products comprising a liquid or with packaging including metal foil at a periphery of the product support grid or in a row of compartments below the RFID reader.

The product support grid may be configured to tilt each compartment except the RFID reader compartment at an angle relative to a vertical direction to tilt the front side of the boxes at least 5° relative to the vertical direction so gravity assists moving first aid product toward the front side of the boxes.

A requisition system including a disclosed first aid cabinet may include a back end computer system including memory where cabinet information regarding each first aid cabinet is stored, the cabinet information including a unique serial number for each first aid cabinet and a cabinet inventory for each first aid cabinet, the cabinet inventory showing first aid products included in the first aid cabinet when the first aid cabinet is fully stocked. The RFID reader of the first aid cabinet collects inventory information by reading all the RFID tags in the first aid cabinet, said RFID reader transmits said inventory information and the unique serial number of the first aid cabinet to the back end. The requisition system back end uses the unique serial number to identify cabinet information corresponding to the first aid cabinet. The back end compares the inventory information to the cabinet inventory to identify first aid products in the cabinet inventory that are not in the inventory information. The requisition system may be configured to automatically generate a restocking requisition for the first aid product associated with each compliance card that has been removed from the enclosure. The inventory information may include an expiration date for some of the first aid products, and the requisition system may be configured to automatically generate a restocking requisition for expired first aid products.

### BRIEF DESCRIPTION OF THE DRAWINGS

FIG. 1 is a front perspective view of a first aid cabinet including an RFID reader connected to an electronic requisition system according to aspects of the present disclosure;

FIG. 2 is an exploded front perspective view of the first aid cabinet of FIG. 1;

FIG. 3 is an exploded rear perspective view of an alternative embodiment of a first aid cabinet enclosure and product support grid according to aspects of the disclosure;

FIG. 4 is a perspective view of an RFID reader and communications enclosure for use in the disclosed first aid cabinets;

FIG. 5 is a side sectional view through the first aid cabinet of FIG. 3;

FIG. 6 is a front view of a first aid cabinet, with the door of the first aid cabinet open showing the product support grid filled with first aid products and including an RFID reader according to aspects of the disclosure;

FIG. 7 is a perspective view of a box of first aid products equipped with a compliance card with angled RFID support portion according to aspects of the disclosure;

FIG. 8 is a perspective view of the compliance card with angled RFID support portion according to aspects of the disclosure removed from the box of first aid products of FIG. 7;

FIGS. 9A-9C illustrate alternative embodiments of a compliance card with angled RFID support portion according to aspects of the disclosure;

FIG. 10 is a side sectional view of a product support grid with angled product compartments according to aspects of the disclosure;

FIG. 11 is a side sectional schematic representation of a box of first aid products incorporating a further embodiment of a compliance card with offset RFID support portion according to aspects of the disclosure;

FIG. 12 is a front perspective view of an embodiment of a compliance card for use in the box of FIG. 11;

FIGS. 13 and 14 are front and rear plan views of the compliance card of FIG. 12 before folding into the shape shown in FIGS. 11 and 12;

FIG. 15 is a front perspective view of a second alternative embodiment of a compliance card of FIGS. 12-14; and

FIG. 16 is a front perspective view of a third alternative embodiment of a compliance card of FIGS. 12-14.

### DETAILED DESCRIPTION

Embodiments of a first aid cabinet and electronic requisition system will now be described with reference to the accompanying Figures, wherein like numerals refer to like or similar elements throughout. Although several embodiments, examples and illustrations are disclosed below, it will be understood by those of ordinary skill in the art that the inventions described herein extend beyond the specifically disclosed embodiments, examples and illustrations, and can include other uses of the inventions and obvious modifications and equivalents thereof. The terminology used in the description presented herein is not intended to be interpreted in any limited or restrictive manner simply because it is being used in conjunction with a detailed description of certain specific embodiments of the inventions. In addition, embodiments of the inventions can comprise several novel features and no single feature is solely responsible for its desirable attributes or is essential to practicing the inventions herein described.

Certain terminology may be used in the following description for the purpose of reference only and thus are not intended to be limiting. For example, terms such as “above” and “below” refer to directions in the drawings to which reference is made. Terms such as “front,” “back,” “left,” “right,” “rear,” and “side” describe the orientation and/or location of portions of the components or elements within a consistent but arbitrary frame of reference which is made clear by reference to the text and the associated drawings describing the components or elements under discussion. Moreover, terms such as “first,” “second,” “third,” and so on may be used to describe separate components. Such terminology may include the words specifically mentioned above, derivatives thereof, and words of similar import.

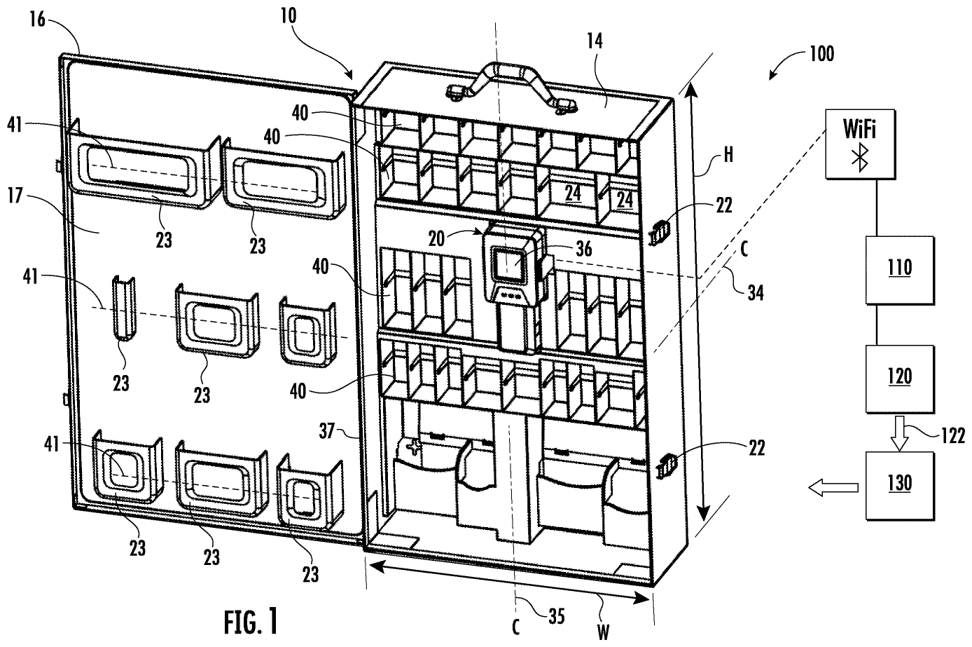

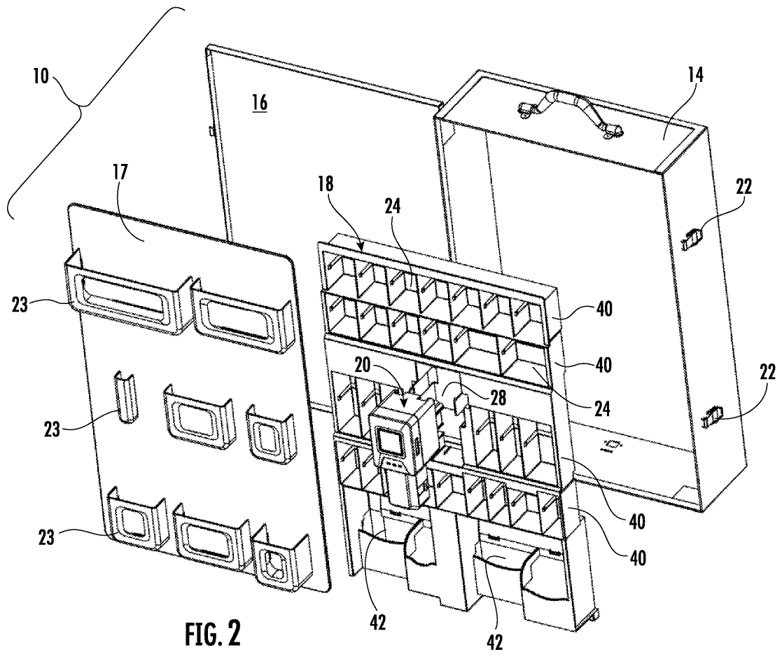

FIGS. 1 and 2 illustrate an embodiment of a first aid cabinet 10 configured for automated RFID inventory of first aid products 12 in the first aid cabinet 10 according to aspects of the disclosure. The first aid cabinet 10 is part of an electronic requisition system 100 configured to manage restocking of first aid cabinets throughout one or more facilities. One version of an electronic requisition system 100 compatible with the disclosed first aid cabinet is described in U.S. Pat. No. 12,008,630, which is assigned to the owner of the present disclosure. The RFID reader 20 is in wired or wireless communication with a user portal 110 and/or directly communicates with a back end 120 of the electronic requisition system 100. The user portal 110 allows the customer to see and manage first aid cabinets 10 at one or more facilities. Alternatively, the first aid cabinets 10 can be managed from the back end 120 electronic requisition system 100, depending on the preference of the customer. In some embodiments, each first aid cabinet 10 has a unique serial number that is stored in the RFID reader 20 and used to identify the first aid cabinet 10 when it communicates with the user portal 110 or the back end 120 of the electronic requisition system 100. The electronic requisition system maintains a record of each first aid cabinet 10 and an inventory of first aid products for each first aid cabinet 10. As previously discussed, first aid cabinets for different types of facilities or departments within a facility will include different first aid products, so each first aid cabinet 10 is managed separately. As discussed in greater detail below, the RFID reader 20 periodically reads all RFID tags associated with first aid products in the first aid cabinet 10 and provides an inventory to the electronic requisition system 100. The electronic requisition system 100 may be configured to automatically generate a requisition 122 for first aid products needed to resupply the first aid cabinet 10. The requisitioned products are used to fill an order 130 that is shipped to the customer facility and used to re-stock the first aid cabinet 10.

A first aid cabinet 10 according to aspects of the disclosure employs an RFID reader 20 that is mounted within an enclosure 14 of the first aid cabinet 10. The RFID reader 20, as will be described below, functions to obtain first aid supplies inventory data at the cabinet 10 and communicates the data via a customer specific application programming interface (API) key to a portal 110 of the electronic requisition system 100, or directly to a back end 120 of the electronic requisition system 100. The back end 120 of the electronic requisition system includes databases storing information about first aid cabinets 10, programs that manage routing of requisitions based on customer preferences, and an API layer for enabling communication with portals 110 and/or first aid cabinets 10. The electronic requisition system 100 provides for customer managed device access, as well as customer-controlled access to the device settings and configuration. The cabinet electronic requisition system 100 integrates with and complements pre-existing inventory techniques, order processing and overall management to provide a system for ensuring the safety and integrity of the first aid cabinet 10. The electronic requisition system 100 provides a secure RFID reader 20 which integrates with the cabinet electronic requisition system 100 to provide centralized management and tracking and to allow a customer to incorporate disclosed first aid cabinets into the requisition system 100 and automate inventory control tasks to ensure first aid cabinets remain fully stocked and compliant with state and federal requirements.

With reference to FIGS. 1 and 2, a metal enclosure 14 includes a door 16 configured to close a front opening of the enclosure 14 to define an interior space for a product support grid 18 and RFID reader 20. The material of the enclosure 14 is not limited to metal, but metal is preferred for its durability. Alternative materials such as plastic may be used. Latches 22 secure the door 16 in the closed position and allow access to the interior of the enclosure 14 when first aid products 12 are needed. The enclosure 14 can be mounted to a wall, pole, or other structure (not shown) in a workplace where access to first aid supplies is needed. The product support grid 18 is configured to fill a rear portion of the interior of the enclosure 14. The product support grid 18 is constructed of non-metallic material such as plastic and defines compartments 24 in which boxes 26, bags, or containers of first aid products 12 are placed to stock the first aid cabinet 10. The compartments 24 are arranged in rows 40. The product support grid 18 also defines an RFID reader compartment 28. The RFID reader 20 includes a housing 30 supporting an RFID antenna 36 and electronics for control and communication. The housing 30 of the RFID reader 20 may be configured to releasably snap into or engage with the RFID reader compartment 28. The antenna 36 of the RFID reader 20 generates RF electromagnetic signals to periodically interrogate RFID tags 32 associated with the first aid products 12 in the enclosure 14.

The first aid cabinet 10 of FIGS. 1 and 2 includes a door panel 17 affixed to the inside surface of the door 16. The door panel 17 supports pockets 23 for additional first aid products. The pockets 23 are arranged in rows 41 and positioned to occupy space at the front of the enclosure 14 when the door 16 is closed. The door panel 17 and pockets 23 add storage for first aid products such as scissors, tweezers or the like that may not be suitable for placement in the product support grid 18. Each package or first aid product in the pockets 23 includes an RFID tag 32.

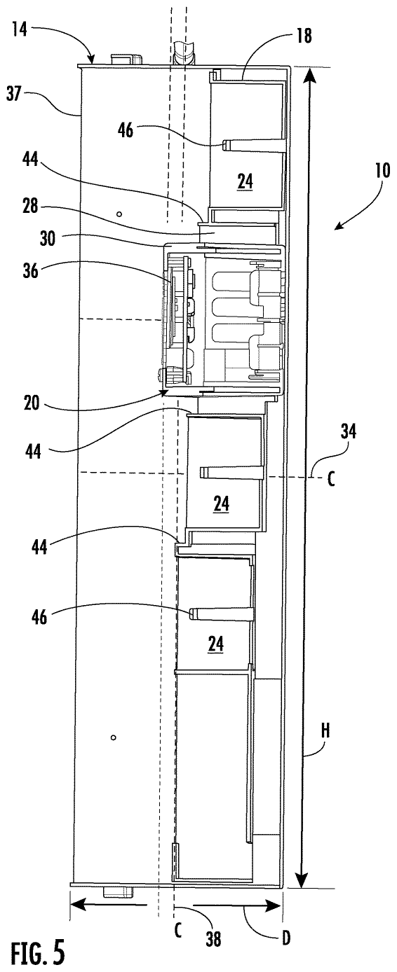

According to aspects of the disclosure, in one embodiment of the product support grid 18, the RFID compartment 28 is located above or below a center position 34 along a vertical height H of the enclosure 14 and aligned with a center 35 of a horizontal width W of the enclosure 14. In this embodiment, the RFID compartment 28 is between 10% and 20% of the height H above or below the center position 34 along the vertical height H. As shown in the first aid cabinet 10 illustrated in FIG. 5, the RFID reader compartment 28 and RFID reader housing 30 are configured to position an RFID antenna 36 forward of a center 38 along the depth D of the enclosure approximately 10% to 20% of the depth D of the enclosure. The RFID antenna 36 is also spaced from the door 16 (and the peripheral lip 37) of the enclosure 14 between approximately 10% and 25% of the depth D of the enclosure 14. This position of the RFID antenna 36 has proven to ensure accurate reading of the RFID tags 32 included in each box 26 of first aid product 12 in the product support grid 18 and RFID tags 32 on first aid products 12 in bins 42 at the bottom of the product support grid 18 and in pockets 23 on the door panel 17.

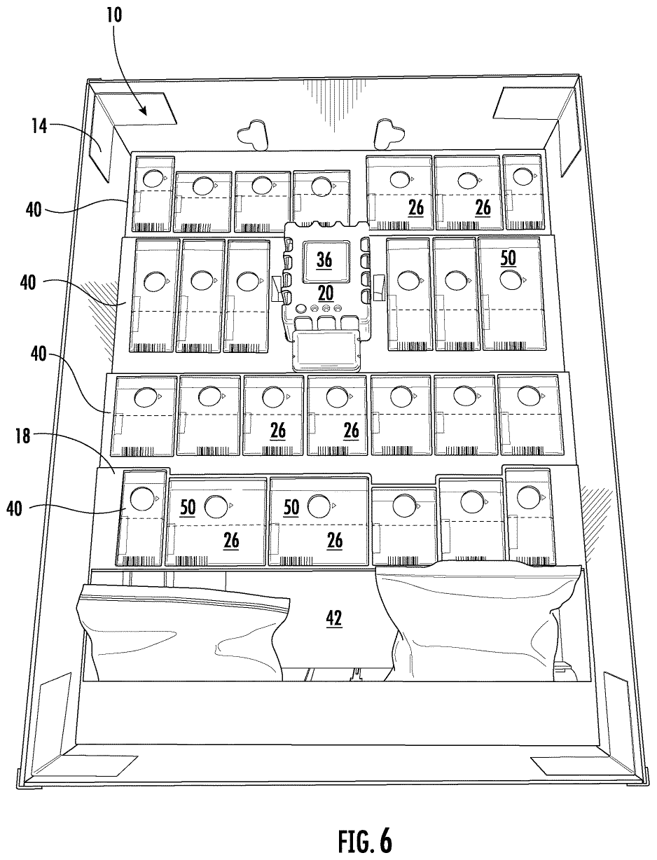

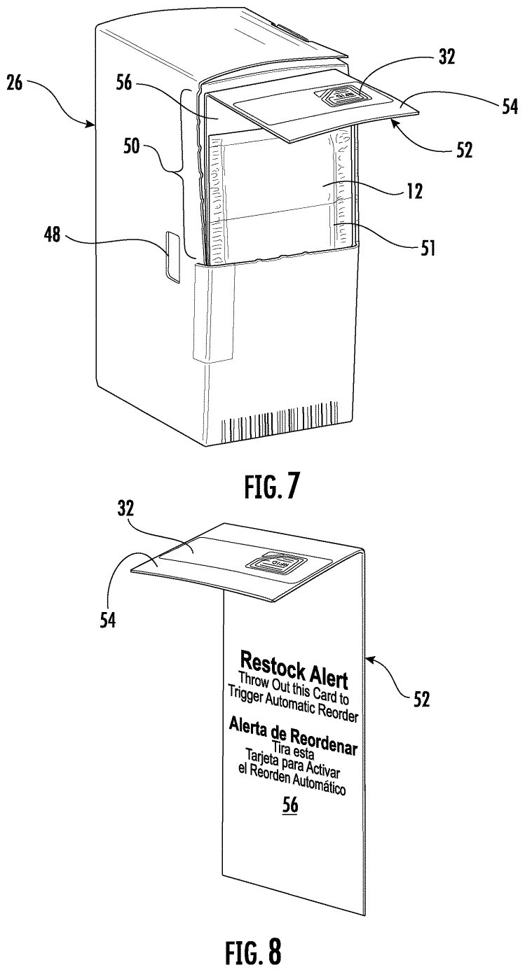

The embodiment of the product support grid 18 illustrated in FIGS. 1 and 2 defines four rows 40 of compartments 24 for boxes 26 of first aid products 12, with a bottom portion of the product support grid 18 including bins or trays 42 for large or bulky first aid products. Products in the bin or tray 42 may be placed in plastic bags or other enclosures and provided with an RFID tag 32 on or in the bag or enclosure (not shown). As best shown in FIGS. 5 and 6, one disclosed embodiment of the product support grid 18 positions all the boxes 26 of first aid products 12 behind the center 38 of the depth D of the enclosure 14. According to aspects of the disclosure, the rows 40 of compartments 24 are offset from each other within the depth D of the enclosure 14, but an offset between rows 40 is not required. As shown in FIG. 5, in one configuration of a product support grid 18 a rear of the top row 40 of compartments 24 are positioned against a back wall of the enclosure 14, and each row 40 below the top row is offset forward in the enclosure 14 relative to the row 40 above it. In the embodiment of a product support grid 18 shown in FIG. 5, the offset 44 between rows 40 is approximately 0.25″ or approximately 6.5 mm. The resulting cascaded configuration of rows 40 of compartments 24 may prevent first aid products 12 in boxes 26 in one row 40 from shielding or blocking RFID tags 32 in boxes 26 in another row 40 from the RFID antenna 36, which can prevent reading all the RFID tags 32 in the enclosure 14. Each compartment 24 is configured to receive a box 26 of first aid product 12. Each row 40 may have a height different from the other rows 40 and each row may define compartments 24 with different widths. According to aspects of the disclosure, each compartment 24 may include tabs 46 that snap into openings 48 on the sides of each box 26 (see FIG. 7) of first aid product 12 to retain the box 26 in the compartment 24. An upper front portion 50 of each box 26 is perforated and can be removed as shown in FIG. 7 to define an opening 51 through which individual first aid products 12 can be removed from the box 26 as needed. Other containers such as a plastic box (not shown) can be configured with a removable portion to allow first aid products to be removed from the container in a similar manner as a paperboard box.

Each first aid cabinet has a unique serial number stored in memory of the RFID reader 20. The cabinet serial number is associated with an inventory stored in the back end 120 of the electronic requisition system 100. The inventory for each first aid cabinet 10 also shows where each box 26 or container of first aid product 12 should be placed in the product support grid 18, pocket 23, bin or tray 42.

According to aspects of the disclosure, a compliance card 52 is included in each box 26 of first aid product 12, with a predetermined number of the first aid product 12 included in each box 26 and a reserve portion of the first aid product 12 behind the compliance card. For example, a box of 20 bandages might have the compliance card 52 positioned to separate a second (reserve) portion of 5 bandages behind the compliance card 52 from a first (primary) portion of 15 bandages in front of the compliance card 52. When the first portion of bandages are depleted, the compliance card is exposed through the opening 51. The compliance card 52 may be brightly colored and includes instructions to remove and dispose of the card to trigger automatic reorder of the first aid product 12 in the box 26. Since the RFID tag 32 is connected to the compliance card 52, removal of the compliance card also removes the RFID tag from the enclosure 14. This method ensures a reserve portion of the first aid product 12 remains in the box 26 of first aid product 12 while an order is placed to restock the first aid product 12. The number of first aid products 12 in a box 26, the primary portion of first aid product in front of the compliance card 52 and the reserve portion of first aid product behind the compliance card 52 can be determined according to the type of first aid product 12 and its expected rate of use. For first aid products 12 expected to have a high rate of use, more than one box 26 of the first aid product 12 may be included in the enclosure 14.

Each compliance card 52 includes an RFID tag 32 loaded with information about the box 26 of first aid product 12. Information on the RFID tag 32 may include the product type, quantity, expiration date, and lot number, but is not limited to this information. According to aspects of the disclosure, the RFID tag 32 is positioned on an RFID support portion 54 that is angled or offset relative to a divider portion 56 of the compliance card 52 to position the RFID tag 32 between the first aid products 12 and the top, bottom, sides, back, or front of the box 26. The divider portion 56 separates first aid products 12 in front of the compliance card 52 from first aid products 12 behind the compliance card 52 to determine a quantity of first aid products 12 remaining in the box 26 as a reserve portion when the compliance card 52 is removed to trigger reorder of the first aid product 12. In one embodiment, the compliance card is folded into a “7” shape as shown in FIGS. 7 and 8, with the RFID support portion 54 positioning the RFID tag 32 above the first aid products 12 and beneath a top of the box 26. The packaging for some first aid products 12 include metal foil which can interfere with the RF signals used to interrogate the RFID tags 32. Other first aid products 12 such as burn cream include liquids that can also interfere with RF signals. Positioning the RFID tag 32 outside of the stack of first aid products 12 in each box 26 ensures the RFID tag 32 will be read every time the RFID reader 20 takes an inventory of the products in the first aid cabinet 10.

According to aspects of the disclosure, the layout of the product support grid 18 and the arrangement of boxes 26 of first aid products 12 in the product support grid 18 can be used to minimize interference with reading RFID tags 32 in boxes 26 of first aid product within the enclosure 14. For example, first aid products containing or including liquids can be positioned in a row 40 below the RFID reader 20. Products with foil packaging or liquids can be strategically positioned within the first aid cabinet 10 so the foil packaging and/or liquid contents do not interfere with reading RFID tags 32 of other boxes 26 of first aid products 12 in the first aid cabinet 10. In another example, first aid products 12 in foil or including liquids can be positioned at the periphery of the product support grid 18 to reduce the likelihood of the foil or liquids coming between the RFID reader 20 and the RFID tags 32 in other boxes 26 of first aid products 12 in the enclosure 14.

As shown in FIG. 1, the vertical position of rows 40 of compartments 24 in the product support grid 18 may be offset from the vertical position of rows 41 of pockets 23 on the door panel 17. This arrangement helps to ensure accurate reading of all RFID tags 32 associated with first aid products on the door panel 17 and in the product support grid 18.

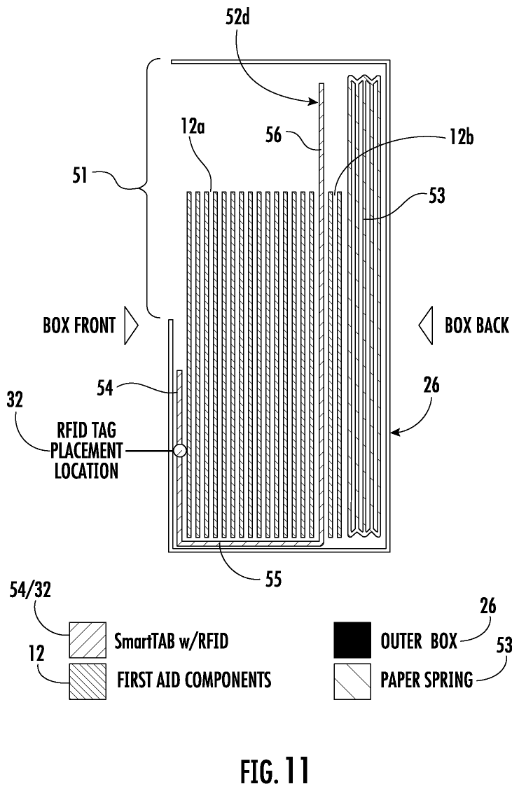

A folded cardboard “spring” 53 of resilient cardboard (see FIG. 11) may be placed behind the first aid products 12 in each box 26 to bias the products 12 forward in the box 26 toward the front opening 51 so as each product 12 is removed, the next product is at the opening 51 as shown in FIG. 7. The forward bias also advances the compliance card 52 toward the opening 51, so the compliance card 52 is clearly visible when the last first aid product 12 in front of the compliance card 52 is removed. According to aspects of the disclosure, the front side of the divider portion 56 of the compliance card 52 may be brightly colored and include simple instructions to remove and dispose of the compliance card 52 when the last product 12 in front of the compliance card 52 has been used. FIG. 10 illustrates an alternative product support grid 18a with compartments 24a angled or tilted to allow gravity to move first aid products 12 and compliance cards 52 in the boxes 26 toward the front opening 51. The product support grid 18a of FIG. 10 illustrates compartments 24a at an angle 58 of approximately 17° relative to the vertical direction or horizontal direction. Compartments 24a may be angled from 5° to 30° to provide a sufficient forward bias to move first aid products 12 to the front of the box 26 as first aid products 12 are used and removed. A forward bias also helps prevent the first aid products 12 from falling away from the opening 51 when fewer first aid products 12 are present in the box 26. The angled compartments 24a shown in FIG. 10 may be used in combination with a cardboard spring 53 or other bias member behind the first aid products 12 to ensure the first aid products 12 advance to the front opening 51 as the products are consumed and to ensure the first aid products 12 do not fall rearwardly in the box 26.

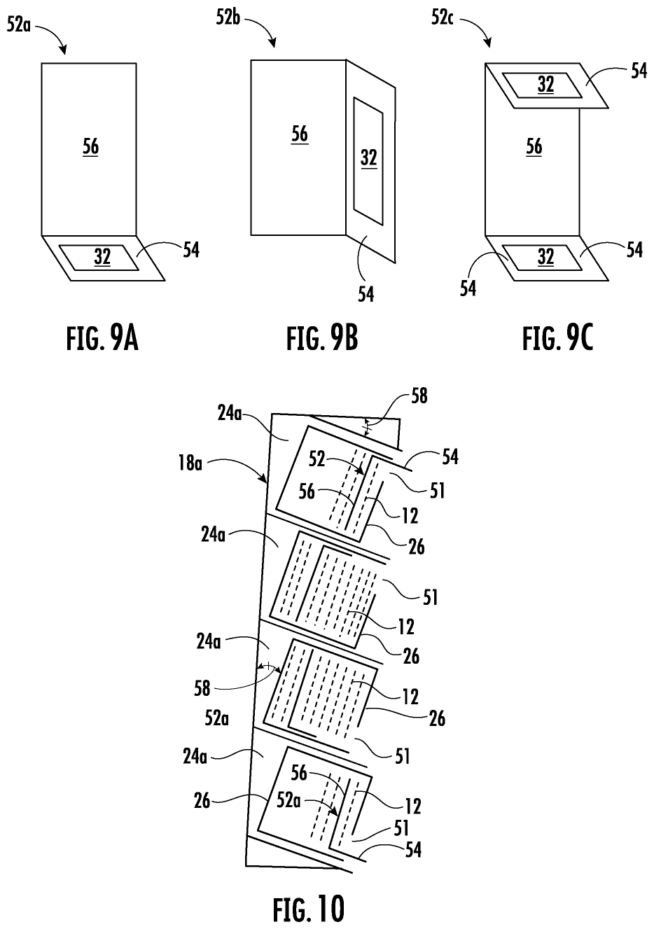

FIGS. 9A, 9B and 9C illustrate alternative configurations of a compliance card 52 according to aspects of the disclosure. FIG. 9A illustrates a compliance card 52a having a divider portion 56 configured to be positioned vertically in a box 26 of first aid products 12 and an angled RFID support portion 54 configured to position an RFID tag 32 beneath the first aid products and between the first aid products and the bottom of a box 26. This form of compliance card 52a may be described as an “L” shape in contrast to the “7” shape illustrated in FIGS. 7 and 8. The bottom two compartments 24a of FIG. 10 illustrate boxes 26 of first aid product 12 configured with a front opening 51 at the bottom of the front of the box 26, allowing the compliance card 52a to advance along the bottom of the box 26 as first aid products 12 are removed from the box 26. FIG. 9B illustrates a compliance card 52b with an angled RFID support portion 54 configured to position the RFID tag 32 along a side of a box of first aid product, between the side of the box and the first aid product. The compliance card 52b of FIG. 9B could be positioned with the RFID support portion 54 on the left or right side of the box 26 so the RFID tag 32 can be positioned within the enclosure 14 at a location spaced apart from the side walls of the enclosure 14 to avoid interference between the RFID tag 32 and the metal wall of the enclosure 14. FIG. 9C illustrates a compliance card 52c with RFID support portions 54 at both the top and bottom of the divider portion 56. RFID tags 32 may be arranged on one or both of the RFID support portions 54 to ensure at least one of the RFID tags 32 is read every time an inventory is taken, whether the box 26 is installed right side up or upside down.

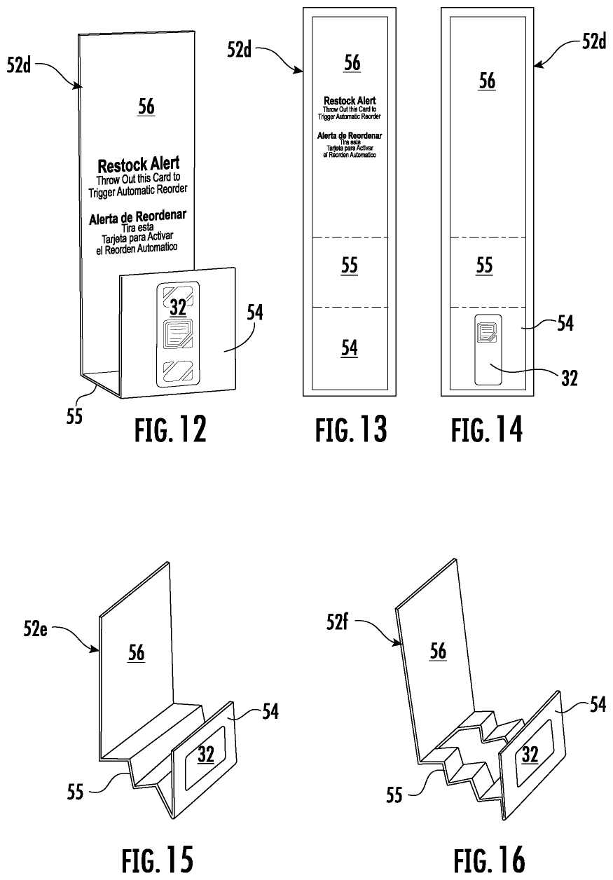

FIG. 11 illustrates a box 26 of first aid products 12 incorporating a further alternative embodiment of a “J” shaped compliance card 52d configured to position the RFID tag 32 between the first aid products 12 and a front of the box 26. This position for the RFID tag 32 facilitates reading by the RFID reader 20 by positioning the RFID tag 32 where there is a minimal likelihood of obstruction by first aid products 12 in the box 26 or first aid products 12 in other boxes within the enclosure 14. As shown in FIG. 11, the RFID support portion 54 of the compliance card 52d is always at the front of the box 26, between the first aid products 12 and the front of the box, regardless of the number of first aid products 12 in the box 26. As shown in FIGS. 12-14, compliance card 52d includes a divider portion 56, an RFID support portion 54, and a connector portion 55 connecting the RFID support portion 54 to the divider portion 56. In this embodiment of a compliance card 52d, the RFID support portion 54 positions the RFID tag 32 parallel to and offset from the divider portion 56, rather than the RFID support portion 54 being angled relative to the divider portion 56 as shown in FIGS. 8 and 9A-9C. According to aspects of the disclosure, at least the front of the divider portion 56 of the compliance card 52d may be brightly colored and include simple, easily understood directions to remove the compliance card 52d to initiate an automatic reorder of the first aid products 12 when the primary portion 12a of first aid products 12 is depleted to expose the front side of the divider portion 56. When the compliance card 52d is removed, the reserve portion 12b of first aid products 12 remains in the box 26. The connector portion 55 is arranged beneath the primary portion 12a of first aid products 12 in front of the divider portion 56 and connects the divider portion 56 to the RFID support portion 54 so the RFID tag 32 is removed along with the compliance card 52d.

FIG. 15 illustrates an alternative “J” shaped compliance card 52e, where the connector portion 55 is folded in a direction parallel to the divider portion 56 and the RFID support portion 54. The accordion folded connector portion 55 allows the divider portion 56 to advance toward the front of the box 26 under the bias of the carboard spring 53 as first aid products 12 are removed from in front of the divider portion 56. FIG. 16 illustrates a further alternative “J” shaped compliance card 52f where the connector portion 55 is weakened by removal of material at the center of the connector portion 55, with the remainder of the connector portion 55 accordion folded to allow the divider portion 56 to advance toward the front of the box 26 as first aid products 12 are removed from in front of the divider portion 56. The connector portion configurations of compliance card embodiments 52e and 52f allow the connector portion 55 to compress or collapse so interference with movement of the divider portion 56 toward the front of the box is minimized. The connector portions 55 of compliance cards 52d, 52e, and 52f ensure the RFID support portion 54 and RFID tag 32 are removed from the box 26 along with the compliance card to initiate an automatic reorder of the first aid products 12 contained in the box 26.

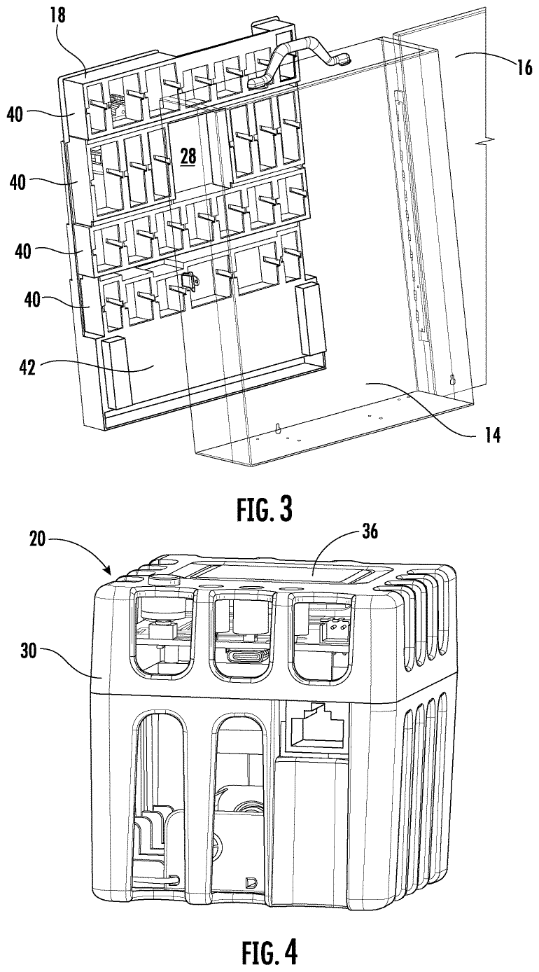

The disclosed first aid cabinet 10 is part of an electronic requisition system 100 configured to automate some steps required to inventory and restock first aid cabinets throughout one or more facilities. One version of an electronic requisition system compatible with the disclosed first aid cabinet 10 is described in U.S. Pat. No. 12,008,630, which is assigned to the assignee of the present disclosure. The RFID reader 20 shown in FIG. 4 includes hardware for wireless (WiFi, Bluetooth) and/or wired (ethernet, serial) communication with an electronic requisition system 100. The RFID reader 20 also includes memory, at least one processor and instructions stored in memory for operation of the RFID reader in the context of the electronic requisition system 100. The RFID reader 20 stores a unique serial number for the first aid cabinet 10, which is used to identify communications from the first aid cabinet 10. The RFID reader 20 includes electrical connectors shown in FIG. 4 for receiving electrical power and for wired communication with the electronic requisition system. The uniquely identified first aid cabinet 10 is associated with an inventory or list of first aid products appropriate for the location of the first aid cabinet. For example, a manufacturing business might have a machine shop and a chemical storage area. A first aid cabinet 10 in the machine shop will be stocked with an inventory of first aid products 12 which may be different from the inventory of first aid products 12 for a first aid cabinet 10 in the chemical storage area, so it is important for the electronic requisition system to be able to identify specific cabinets within the system.

According to aspects of the disclosure, instructions for interrogating all the RFID tags 32 in the first aid cabinet 10 on a schedule selected by personnel responsible for managing the first aid cabinets may be stored locally in the memory of the RFID reader 20, or the RFID reader 20 may be configured to respond to a command from the electronic requisition system 100 via wired or wireless communication to initiate an inventory. For example, the RFID reader 20 can be programmed to take an inventory of the first aid cabinet 10 at a particular time every day. The RFID reader 20 receives a response from each of the RFID tags 32 in the first aid cabinet 10, which is compared to a list of first aid products 12 for the first aid cabinet 10 in the back end 120 of the electronic requisition system 100. A missing RFID tag 32 triggers an automatic order to replenish the first aid product 12 associated with the missing RFID tag 32. The inventory can also be used to track the expiration dates of first aid products 12 within the first aid cabinet 10. When an inventory shows a box 26 of first aid products is approaching or has passed its expiration date, the electronic requisition system 100 can be programmed to automatically generate an order for the expired first aid products 12.

Instructions for each inventory of the first aid cabinet 10 may include interrogating the RFID tags 32 in the first aid cabinet more than once. If the same RFID tags 32 are read during each interrogation, then the inventory can be accepted as correct. If two or more inventories produce a different list of RFID tags 32, an error might be reported. Another round of interrogations can be conducted and if no error is detected on this round, then the inventory can be accepted as correct. If errors repeatedly occur, the system can be configured to report a problem with the first aid cabinet 10 requiring personnel to visit the cabinet to remedy the issue. A visit to a first aid cabinet 10 might also be called for if an inventory produces a list of first aid products not matching the list of first aid products for the particular first aid cabinet. Since personnel are responsible for restocking the first aid cabinet, it is possible for boxes or containers of an incorrect first aid product to be placed in the cabinet and the electronic requisition system can be configured to send an alert for the error to be corrected.

It should be emphasized that many variations and modifications may be made to the herein-described embodiments, the elements of which are to be understood as being among other acceptable examples. All such modifications and variations are intended to be included herein within the scope of this disclosure and protected by the following claims. Moreover, any of the steps described herein can be performed simultaneously or in an order different from the steps as ordered herein. Moreover, as should be apparent, the features and attributes of the specific embodiments disclosed herein may be combined in different ways to form additional embodiments, all of which fall within the scope of the present disclosure.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment.

What is claimed:

1. A first aid cabinet comprising:
an enclosure defining an interior having a height H, a width W and a depth D, the enclosure including a door and a latch to secure the door in a closed position;
a product support grid, the product support grid defining a plurality of rows of compartments and an RFID reader compartment, said RFID reader compartment positioned between 10% and 20% of the height H above or below a center of the height H of the enclosure;
an RFID reader positioned in the RFID reader compartment, said RFID reader including an RFID antenna;
a box of first aid product in a plurality of the compartments, each said box including a front side exposed at a front of the product support grid, the front side of each box including a removable portion, each said box including a predetermined quantity of first aid products and a compliance card having a divider portion and an RFID support portion angled or offset relative to the divider portion, an RFID tag on the RFID support portion positioned between first aid products in the box and a top, bottom, front, back, or side of the box, the RFID tag is loaded with information including at least the type of first aid product in each box, the divider portion is arranged between the first aid products to divide the first aid products into a primary portion between the divider portion and the front of the box and a reserve portion between the divider portion and the rear of the box, the divider portion having a front side facing the front of the box,
wherein the removable portion of the box is removed define an opening in the front side of each box to allow dispensing of first aid products through the opening, removal of the primary portion of first aid products exposes the front side of the divider portion, after which the compliance card is removed along with its RFID tag, the RFID reader is configured to periodically read all the RFID tags in the enclosure to generate a list of first aid products associated with compliance cards that remain in the enclosure.

2. The first aid cabinet of claim 1, wherein the front side of the divider portion includes a notice to remove the compliance card when the primary portion of first aid products is depleted to expose the front side of the compliance card.

3. The first aid cabinet of claim 1, wherein the enclosure is metal, or the enclosure is non-metal.

4. The first aid cabinet of claim 1, wherein the RFID antenna is spaced apart from the door of the enclosure a distance between approximately 10% and 25% of the depth D of the enclosure when the door is in a closed position.

5. The first aid cabinet of claim 1, wherein each row of compartments is offset within the depth D of the enclosure relative to the other rows of compartments.

6. The first aid cabinet of claim 1, wherein the RFID antenna is positioned forward of a center of the depth D of the enclosure.

7. The first aid cabinet of claim 1, wherein the first aid products in the first aid cabinet include first aid products comprising a liquid or packaged in packaging including metal foil, said product support grid configured to position boxes of first aid products comprising a liquid or packaged in packaging including metal foil at a periphery of the product support grid or in a row of compartments below the RFID reader.

8. The first aid cabinet of claim 1, wherein each compartment except the RFID reader compartment is angled relative to a vertical direction to tilt the front side of the boxes between 5° and 30° relative to a vertical direction.

9. A box of first aid product comprising:
a bottom, top, front, back, left and right sides defining a container, the front side including a removable portion, removal of the removable portion defining an opening;
a predetermined quantity of first aid products inside the container and removable through the opening; and
a compliance card having a divider portion and an RFID support portion angled or offset relative to the divider portion, an RFID tag on the RFID support portion positioned between the first aid products and a top, bottom, or side of the box, the RFID tag loaded with information including at least the type of first aid product, the divider portion positioned between the first aid products to divide the predetermined quantity of first aid products into a primary portion between the divider portion and the front side of the container and a reserve portion between the divider portion and the rear side of the container, the divider portion having a front side facing the front of the container,
wherein the removable portion of the box is removed define an opening in the front side of each box to allow dispensing of first aid products through the opening, removal of the primary portion of first aid products exposes the front side of the divider portion, after which the compliance card is removed along with its RFID tag.

10. The box of first aid product of claim 9, wherein the front side of the divider portion includes a notice to remove the compliance card when the primary portion of first aid products is depleted to expose the front side of the compliance card.

11. The box of first aid product of claim 9, wherein the compliance card includes a connector portion connecting the divider portion to the RFID support portion, the RFID support portion arranged between the primary portion of first aid product and the front side of the box.

12. The box of first aid product of claim 9, wherein the connector portion is folded to allow the divider portion to move toward the front side of the container as first aid products are removed from the primary portion.

13. The box of first aid product of claim 9, comprising a resilient member between the reserve portion of first aid product and the back side of the container, said resilient member urging the first aid product toward the front side of the container.

14. A requisition system including the first aid cabinet of claim 1, said requisition system comprising:
a back end including memory where cabinet information regarding each first aid cabinet is stored, the cabinet information including a unique serial number for each first aid cabinet and a cabinet inventory for each first aid cabinet, the cabinet inventory showing first aid products included in the first aid cabinet when the first aid cabinet is fully stocked,
wherein, the RFID reader of the first aid cabinet collects inventory information by reading all the RFID tags in the first aid cabinet, said RFID reader transmits said inventory information and the unique serial number of the first aid cabinet to the back end, said back end using the unique serial number to identify cabinet information corresponding to the first aid cabinet, said back end comparing the inventory information to the cabinet inventory to identify first aid products in the cabinet inventory that are not in the inventory information.

15. The requisition system of claim 14, wherein the requisition system automatically generates a restocking requisition for the first aid product associated with each compliance card that has been removed from the enclosure.

16. The requisition system of claim 14, wherein the inventory information includes an expiration date for some of the first aid products, and the requisition system automatically generates a restocking requisition for expired first aid products.
